(19) United States
(12) Patent Application Publication
(10) Pub. No.: US 2008/0306010 A1
(43) Pub. Date: Dec. 11, 2008

(54) VACCINE COMPOSITION CONTAINING IRRADIATED OVALBUMIN FOR THE PREVENTION AND TREATMENT OF ALLERGIC DISEASE

(75) Inventors: Myung-Woo Byun, Deajeon-Shi (KR); Ju-Woon Lee, Jeollabukdo (KR); Jae-Hun Kim, Jeollabukdo (KR); Jee-Hyun Seo, Deajeon-shi (KR)

Correspondence Address: LUCAS & MERCANTI, LLP, 475 PARK AVENUE SOUTH, 15TH FLOOR, NEW YORK, NY, 10016, US

(73) Assignee: KOREA ATOMIC ENERGY RESEARCH INSTITUTE, Daejeon (KR)

(21) Appl. No.: 11/853,341

(22) Filed: Sep. 11, 2007

(30) Foreign Application Priority Data
Jun. 7, 2007 (KR) 10-2007-55431

(51) Int. Cl. A61K 38/38; C07K 14/77; A61P 37/08; C07K 1/14

(52) U.S. Cl. 514/21; 530/367; 530/368

(57) ABSTRACT

The present invention relates to a vaccine composition for the prevention and treatment of allergic disease comprising irradiated ovalbumin as an effective ingredient, more precisely a method for preparing an immunogen of a vaccine for the prevention and treatment of allergic disease using the irradiated ovalbumin which is separated and purified from the albumen of an egg, a vaccine composition for the prevention and treatment of allergic disease comprising the irradiated ovalbumin as an effective ingredient, and a method for the prevention and treatment of allergic disease using the vaccine comprising the irradiated ovalbumin. In the mouse vaccinated with the irradiated ovalbumin, humoral and cell mediated immune responses were both reduced, suggesting that allergic reaction was inhibited. Thus, the composition of the present invention can be effectively used as a vaccine for the prevention and treatment of allergic disease.

### TECHNICAL FIELD

The present invention relates to a vaccine composition for the prevention and treatment of allergic disease comprising irradiated ovalbumin as an effective ingredient more precisely a method for preparing an immunogen of a vaccine for the prevention and treatment of allergic disease by irradiating ovalbumin which is separated and purified from the albumen of an egg, a vaccine composition for the prevention and treatment of allergic disease comprising the irradiated ovalbumin prepared by the above method, and a method for the prevention and treatment of allergic disease by using the vaccine above.

### BACKGROUND ART

Allergic disease is very common not only in advanced countries but also in Korea and its prevalence rate increases every year. Allergen, that is a causative antigen of allergic disease, is exemplified by dust mite, bee venom, pollen and food, etc. Allergy is the clinical symptom especially shown in those who have an allergen specific IgE antibody. Allergic reaction such as local urticaria, eruption, allergic rhinitis, asthma and systemic reaction like anaphylaxis can cause serious problems and might results in death (Broide D H. J Allergy Clin Immunol 2001; 108:s65-71.

In particular, food allergy is the food specific IgE mediated hypersensitivity reaction. Food allergy has been continuously increased for the past 20 years in USA and Europe and according to a recent report the prevalence rate of adults is now more than 2% and the prevalence rate of children is more than 8% (Sampson H A. J Allergy Clin Immunol 2003; 111:s540-7).

Food allergy is most likely developed in infants and preschool children under the age of 3. More than 90% of those children who have food allergy exhibit allergic reaction against milk, egg, soybean, flour, peanut or fisheries. In the meantime, allergic reaction in adults is mostly against peanut and fisheries. In Korea, allergic reactions against mackerel, peach, pork, milk, egg, buck wheat, crustacea, wheat, pupa and tomato are frequently reported and in fact allergic reaction varies from the diet habit (Kim, et al. Ashma and Allergy 2003; 23: 502-14, Han, et al., Korean J. Food Nut. 1997; 26:1-9).

Most of the clinical symptoms caused by food allergy are detected in the gastrointestinal organs and on skin. Gastrointestinal disease caused by allergy is allergic eosinophilic gastroenteritis exhibiting symptoms like indigestion, diarrhea and vomiting, and skin disease by allergic reaction is exemplified by skin rash, atopic dermatitis, urticaria and angioedema, etc.

Protein in food is decomposed in the gastrointestinal track into smaller molecular weight molecules such as peptides and amino acids, which are absorbed in the intestinal cells. In spite of physiological and immunological barriers against allergen invasion, food allergens can invade the gastrointestinal tracks of infants and children who have the comparatively immature gastrointestinal organs (Metchlfe D D, et al., Blackwell Science, USA, 1997).

Food allergen migrates via M cells (microfold cells) of the intestinal track and further migrates into intestinal mucosa and finally reaches antigen-processing cells such as dendritic cells. The peptides produced by the antigen-processing cells are extracellular expressed along with MHC II (major histocompatibility complex class II) (Mayer L. Am J Physiol 274:G7-9, 1998). The antigen-processing cells activated by the above process stimulate or activate T-cells. By the interaction with the antigen peptide, MHC class II and other co-stimulator signal molecules, type 2 helper T cells (Th2 cells) regulate TL-4 release and IgE synthesis and at the same time produce eosinophils involved in inflammation by IL-5 and IL-13. These cytokines stimulate helper T cells and B cells to produce IgE antibody in B cells. IgE production is a key factor in food allergy development, suggesting that IgE acts as a mediator for food allergy. Once IgE antibody reaches mast cells of the intestinal mucosa or basophils in blood, the IgE antibody molecule adheres to the high-affinity receptor (FcεRI) on the surface of a mast cell to induce degranulation of the mast cell and accelerate the release of a mediator such as histamine (Broide D H. J Allergy Clin Immunol, 108:S65-71, 2001). The inflammation mediator causes allergic reactions when it adheres onto a target organ such as bronchi, skin, eye or gastrointestinal track.

Although food allergy has a potential for causing a serious allergic reaction, no specific treatment has been established so far (Nowak-Wegrzyn A. Inflammation & Allergy-Drug Targets, 5:23-34, 2006). The best way to prevent food allergy known so far is to avoid taking a causative food or processed food containing any causative food. To protect infants from allergy, a woman in childbed is encouraged to avoid a causative food before breast-feeding. However, it is very difficult to avoid a causative food completely and limitation in food-taking might results in retardation in growth of a child or nutritional unbalance. When regulation of a diet was failed or could not control allergic reaction or acute allergic symptoms were developed, medicines such as corticosteroid, anti-histamines and sodium chromolicate had been administered. However, long-term administration of such medicines brings side-effects. Therefore, it is urgent request to work out a fundamental solution for allergic disease.

To reduce side effects during the treatment of allergic disease, immuno-therapy was proposed as an alternative. For an example, allergen immuno-therapy is performed to relieve clinical symptoms by IgE antibody mediated allergy. Particularly, the dose of a specific allergen (allergen extract) increases over every administration to induce immunological tolerance, which has been particularly effective to treat respiratory allergy. Such allergen immuno-therapy induces two different immunological reactions in helper T-cells. That is, Th2 immune response causes the decrease of IgE antibody and cytokines such as IL-4 and Il-5 in serum, and thus induces the increase of IgG2a antibody and another cytokine IFN-γ in serum, which is known to be involved in Th1 immune response, leading to Th1 immune response (WHO. WHO position paper, Geneva, Jan. 27-29, 1997).

Recent reports on immunological mechanisms involved in food allergen and allergic reaction push the advancement of technologies and methods for the prevention and treatment of food allergy. However, a concrete treatment to control food allergy has not been made, yet, even if studies on food allergy started 1900s.

Studies on immuno-therapy for IgE mediated food allergy started 100 years ago. In the meantime, immuno-injection therapy was tried to treat fish allergy and successful desensitization result was reported. However, even if desensitization was successfully induced from the clinical tests, the risk of systemic hypersensitivity reaction was higher, compared with the respiratory allergy, which worried us so that the immuno-injection therapy is not recommended today (Burks A W. Allergy 67:121-4, 2003).

To reduce side effects of immuno-therapy for not only food allergy but also other allergic diseases, new technology using anti-IgE antibody, peptide, recombinant allergen (US Patent No. US 2005/0774464), mutant allergen, probiotics (Korean Patent No. is KR20040068820 and Korean Patent No. KR20060088341), plasmid DNA, oligodeoxynucleotide and Th1 adjuvant, has been tried (Pons L, et al., Curr Opin, Allergy Clin Immunol 5:558-62, 2005; Nowak-Wegrzyn A. Inflammation & Allergy-Drug Targets 5:23-34, 2006: Crameri. R, et al., Curr Opin Immunol, 18:761-8, 2006). In addition, Oriental herbs (WO 2005/092360) or a histamine inhibitor (WO 2006/038656) has been tried to treat allergy.

The above mechanism to treat allergy is targeting the inhibition of histamine that is secreted during the allergic reaction and Th2 response by increasing Th1 immune response. The recent immuno-therapy for allergy uses such allergens as allergen extract, recombinant allergen and physically modified allergen or chemically modified allergen. The recombinant allergen has a very similar structure with allergen, so it is expected as an alternative for standardization of allergen extracts but at the same time there is still a disadvantage of side effects in using this recombinant allergen for immunotherapy. The physically or chemically modified allergen has almost no binding capacity to IgE but still can be reacted with T-cells, suggesting that this method is safer in preventing and treating allergy, compared with the conventional methods. Another attempt is that an allergen: is adhered to an adjuvant such as aluminum hydroxide and then accumulated, by which administration frequency cab be reduced. It has also been reported that an allergen (allergoid) was polymerized (U.S. Pat. No. 5,334,848) by using a chemical reagent such as formaldehyde and glutaraldehyde and this synthetic allergen could inhibit IgE, IgG and IL-4, indexes for allergic reaction, in animal tests (Hayglass K T, Stefura W P. J Immunol 147:2455-60, 1991). A large amount of the allergoid might be administered compared with the original allergen extract, because the allergoid is polymerized. Thus, the allergoid has an advantage of less administration frequency, suggesting that a patient is less required to visit a hospital, but at the same time has a disadvantage of difficulty in preparing a standardized composition thereof by chemical polymerization.

The success of the allergen immuno-therapy is closely related to the production of allergen blocking IgG, relieve of clustering of eosinophils and T-cells, change of immune response pattern from Th2 immune response to Th1 immune response, and/or nullifying the reaction of T-cells (Frew A J. J Allergy Clin Immunol, 111:s 712-9, 2003). Therefore, studies have been focused on the development of an allergen composition or an adjuvant that can induce immune tolerance and Th2 response inhibition.

Food irradiation is used for sprout inhibition, life extension, extermination of parasite and vermin, decay prevention and sterilization of pathogenic microorganisms in food and this technique is particularly to irradiate the energy, that is ionized radiation energy from radioactive materials or radioactive ray generator such as gamma ray (Co-60 or Cs-137), electron beam and X-ray, to food at the radiation dose of 0.01 kGy-200 kGy. Recently, irradiation is used not only for sterilization of food but also for changing the structure of such carcinogenic substance as nitrosamine and biogenic amine, generated during food processing and storage, with reducing its toxicity.

Similar attempts using irradiation have been made to reduce food allergy inducing substances. Irradiation was performed onto eggs (Lee J W, et al., J Food Prot 65:1196-9, 2002), milk (Lee J W, et al., J Food Prot 64:272-6, 2001) and shrimp (Byun M W, et al., J Food Prot 63: 940-4, 2000) which are most representative allergy inducing substances. As a result, the binding capacity of these irradiated allergens to IgE in serum of an allergy patient was significantly reduced. Digestion stability was also investigated using pepsin and trypsin. As a result, allergenicity of ovalbumin that is an allergen of an egg was significantly reduced by the treatment of such digestive enzyme as pepsin or trypsin after irradiation of the egg (Seo J H, et al., J Food Prot 67:1463-8, 2004). Albumen was separated from an egg, which was then irradiated. Bread was produced with the irradiated albumen. As a result, the binding capacity of the egg allergen to IgE in patient's serum was obviously reduced, suggesting that food irradiation is effective to reduce allergen (Lee J W, et al., Radiat Physics Chem 72:645-650, 2005). As explained hereinbefore, the modified allergen by irradiation results in the decrease of allergenicity. An irradiated allergen was intra-abdominally injected in a test animal and changes of immunogenicity were observed. As a result, IgE and other antibody subclass responses were reduced and so were the responses of IL-4 and other cytokines (Seo J H, et al., Int Immunopham. 7:464-72, 2007).

The present inventors irradiated ovalbumin, the most representative allergy inducing substance, to change its structure and completed this invention by confirming that a vaccine comprising “the irradiated ovalbumin” as an effective ingredient can be effectively used for the prevention of allergy.

### BRIEF DESCRIPTION OF DRAWINGS

The application of the preferred embodiments of the present invention is best understood with reference to the accompanying drawings, wherein:

FIG. 1 is a schematic diagram illustrating the allergic inhibitory effect of a vaccine composition for the prevention of allergic disease comprising the irradiated ovalbumin.

FIG. 2 is a graph illustrating the levels of ovalbumin-specific IgGAM and ovalbumin-specific IgE antibody induced with allergic reaction in serum of the mouse vaccinated with the irradiated ovalbumin:

*: p<0.001

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 3 is a graph illustrating the levels of such ovalbumin-specific IgC1, IgG2a, IgG2b, IgG3, IgM and IgA antibodies induced with allergic reaction in serum of the mouse vaccinated with the irradiated ovalbumin:

**: p<0.01;

***: p<0.01;

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 4 is a graph illustrating the levels of Th1 response related cytokines IL-2 and IFN-γ, and the levels of Th2 response related cytokines IL-4 and IL-10 induced with allergic reaction in the mouse immunized with the irradiated ovalbumin:

**: p<0.01;

***: p<0.001;

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 5 is a graph illustrating the levels of T-cell proliferation induced with allergic reaction in the mouse immunized with the irradiated ovalbumin:

*: p<0.001;

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen: cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 6 is a graph illustrating the levels of ovalbumin-specific IgGAM antibody induced with allergic reaction in serum of the mouse that T-cells and B-cells were separated from the spleen of the mouse immunized with the irradiated ovalbumin, and then those separated cells were injected into a mouse not-treated to induce allergic reaction:

*: p<0.05;

**: p<0.01;

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 7 is a graph illustrating the levels of IgE among ovalbumin-specific antibodies in the mouse serum that was gained by immunizing the mouse with the irradiated ovalbumin, dividing the separated spleen cells into T-cells and B-cells, injecting said cells into the vein of a non-treated mouse respectively, and then inducing allergic reaction:

*; p<0.05;

**: p<0.01

***: p<0.0001;

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 8 is a graph illustrating the levels of IgG1 and IgG2a among ovalbumin-specific antibodies in the mouse serum that was gained by immunizing the mouse with the irradiated ovalbumin, dividing the separated spleen cells into T-cells and B-cells, injecting said cells into the vein of a non-treated mouse respectively, and then inducing allergic reaction:

*: p<0.05;

**: p<0.01;

***: p<0.001;

N: normal mouse group without treatment;

I: control group induced with allergic reaction without immunization;

O: spleen cells of the control group induced with allergic reaction after immunizing with non-irradiated ovalbumin,

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

FIG. 9 is a graph illustrating the levels of T-cell proliferation in the mouse serum that was gained by immunizing the mouse with the irradiated ovalbumin dividing the separated spleen cells into T-cells and B-cells, injecting said cells into the vein of a non-treated mouse respectively, and then inducing allergic reaction:

*: p<0.051;

**: p<0.01;

N: normal mouse group without treatment;

I: control group induced with allergic reaction without immunization;

O: spleen cells of the control group induced with allergic reaction after immunizing with non-irradiated ovalbumin;

10: spleen cells of the experimental group induced with allergic reaction after immunizing with irradiated ovalbumin at 10 kGy;

40: spleen cells of the experimental group induced with allergic reaction after immunizing with irradiated ovalbumin at 40 kGy; and

100: spleen cells of the experimental group induced with allergic reaction after immunizing with irradiated ovalbumin at 100 kGy.

### DISCLOSURE

### Technical Problem

It is an object of the present invention to provide a method for preparing an immunogen of a vaccine for the prevention and treatment of allergic disease which has a modified structure by irradiation.

### Technical Solution

To achieve the above object, the present invention provides a method for preparing an immunogen of a vaccine for the prevention and treatment of allergic disease.

The present invention also provides a vaccine composition for the prevention and treatment of allergic disease comprising the irradiated ovalbumin prepared by the above method as an immunogen.

The present invention further provides a method for prevention and treatment of allergic disease by using the above vaccine composition.

Hereinafter, the present invention is described in detail.

The present invention provides a: method for preparing an immunogen of a vaccine for the prevention and treatment of allergic disease, which comprises the following steps:

1) Separating and purifying ovalbumin from an egg; and

2) irradiating the ovalbumin. in aqueous solution.

The ovalbumin is a protein or glycoprotein acting as an allergen causing an allergic reaction in human or animals.

The ovalbumin is preferably obtained by separating albumen and yolk from an egg; diluting the separated albumen in phosphate buffer; and separating and purifying the ovalbumin from the albumen solution by using column. The ovalbumin includes any purified protein as long as it is purified according to the conventional protein purification method.

The aqueous solution in the above step 2) is preferably phosphate buffer but not always limited thereto and in fact any solution including saline that is able to dissolve a protein and is applicable to human can be used.

The radioactive ray used in step 2) is preferably high energy gamma ray, X-ray or electron beam (beta ray). As a radiation source for food irradiation, specifically Co-60 or Cs-137 is preferred and irradiation can be performed by the X-ray generator having up to 5 MeV energy or the electron beam apparatus having up to 10 MeV energy.

The radiation dose of the above radioactive ray is preferably 5-100 kGy and more preferably 40-100 kGy and most preferably 100 kGy, but not always limited thereto and 1 kGy or up is acceptable.

The allergic disease in step 2) is preferably food allergy but not always limited thereto.

The immunogen prepared by the above method is called “irradiated ovalbumin”.

The present invention also provides a vaccine composition for the prevention and treatment of allergic disease comprising the irradiated ovalbumin prepared according to the above method as an immunogen.

The preferable dosage of the irradiated ovalbumin is 0.5-3 μg/g and 1-2 μg/g is more preferable and 1 μg/g is most preferable, but not always limited thereto and the dosage can be determined in the range that can provide preventive and treatment effect on allergic disease.

The immunologically effective dosage of the vaccine composition of the present invention can be determined according to the dosage required for inducing immune response. And the dosage can be determined by those in the art by considering age and weight of a patient, clinical symptoms and the administration method.

It is also preferred for the irradiated ovalbumin to be adhered onto an aluminum hydroxide adjuvant at the ratio of 1:100-200 weight part and the ratio of 1:100-150 weight part is more preferred and the ratio of 1:130 weight part is most preferred, but the ratio can be varied from each disease or symptoms.

The allergic disease above is preferably food allergy but not always limited thereto.

Most allergens are composed: of proteins. Once a protein is irradiated, disulfide bond, hydrogen bond, hydrophobic interaction and ionic bond in the protein are affected, resulting in fragmentation or aggregation with causing secondary or tertiary protein structural changes.

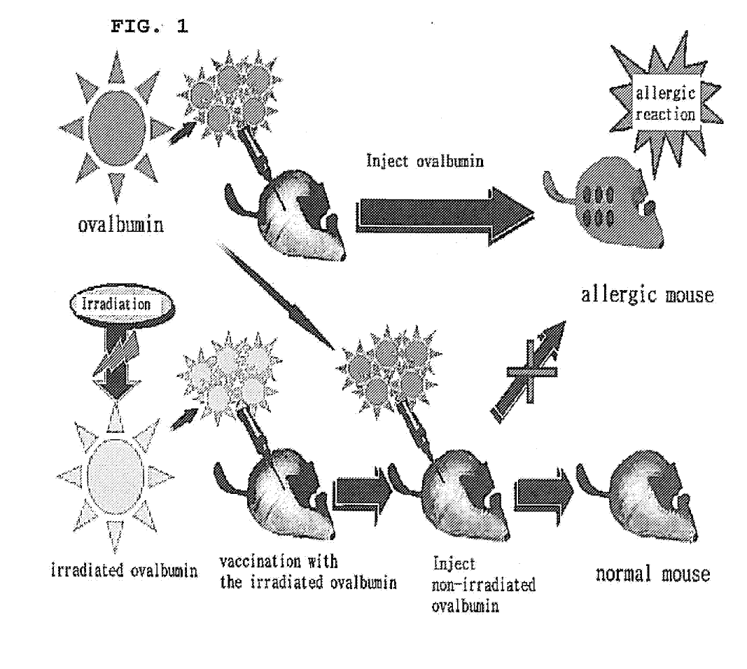

Based on the above principal, the present inventors investigated the effect of the irradiated ovalbumin prepared by the method of the invention, a modified allergen with structural changes by irradiation, on immunological symptoms of test animals (see FIG. 1).

First, mice were vaccinated with the irradiated ovalbumin by intra-abdominal injection to investigate the inhibitory effect of the irradiated ovalbumin on the allergic reaction caused by non-irradiated ovalbumin. And humoral immunity was examined in the mice induced with allergic reaction after vaccination.

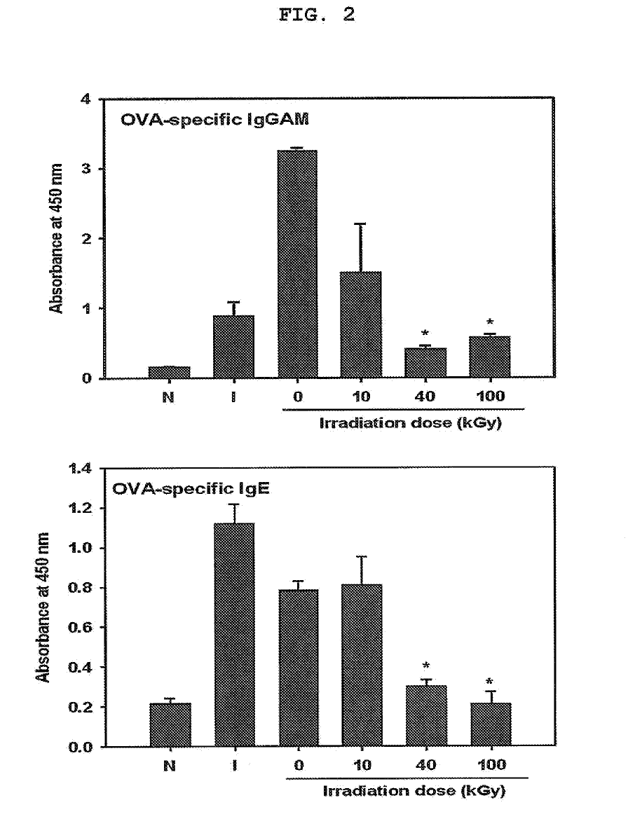

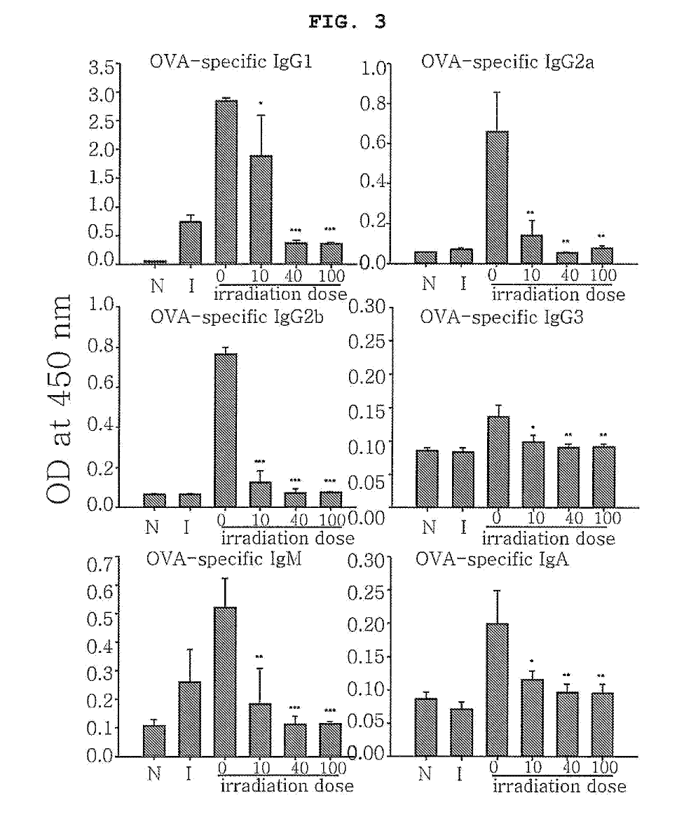

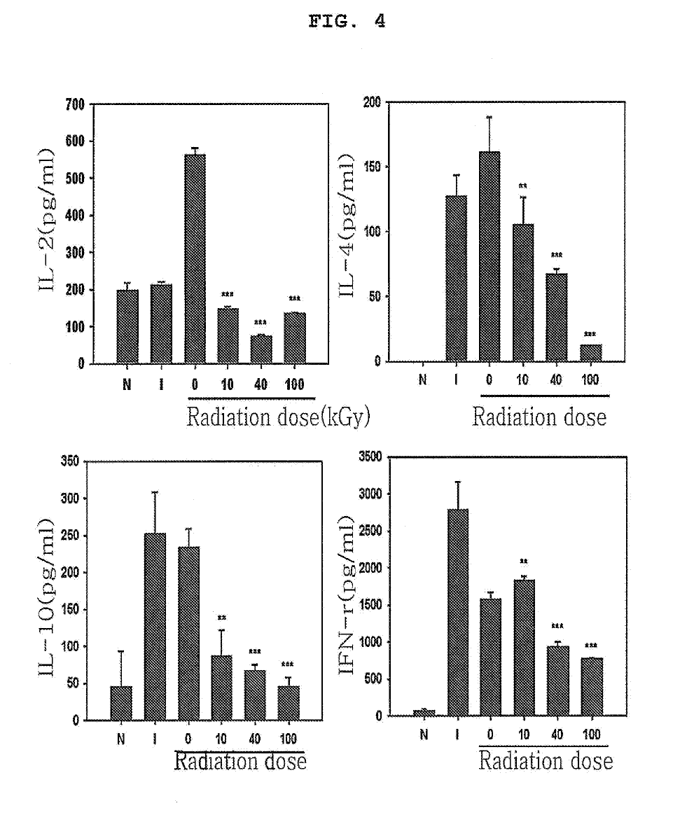

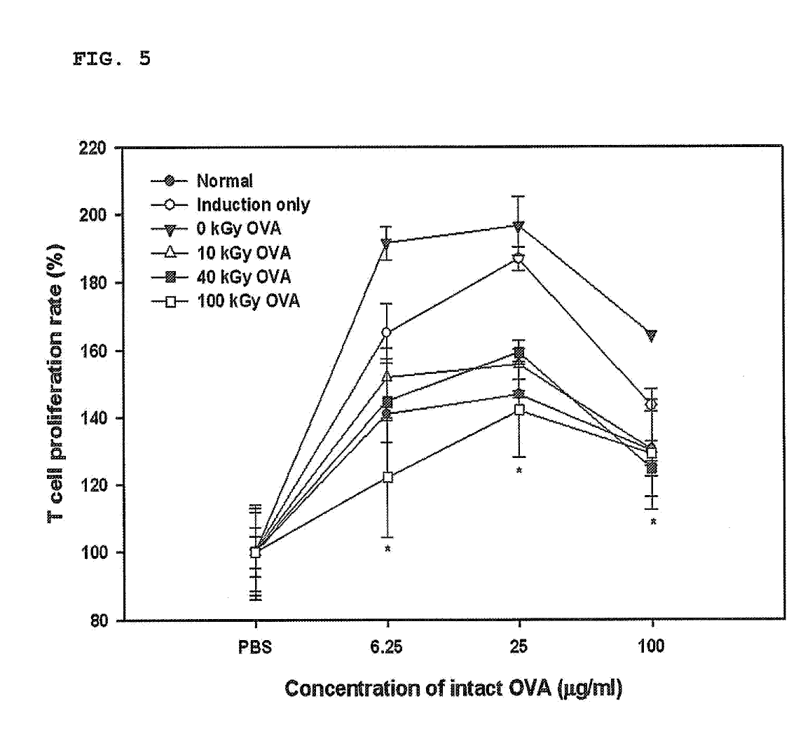

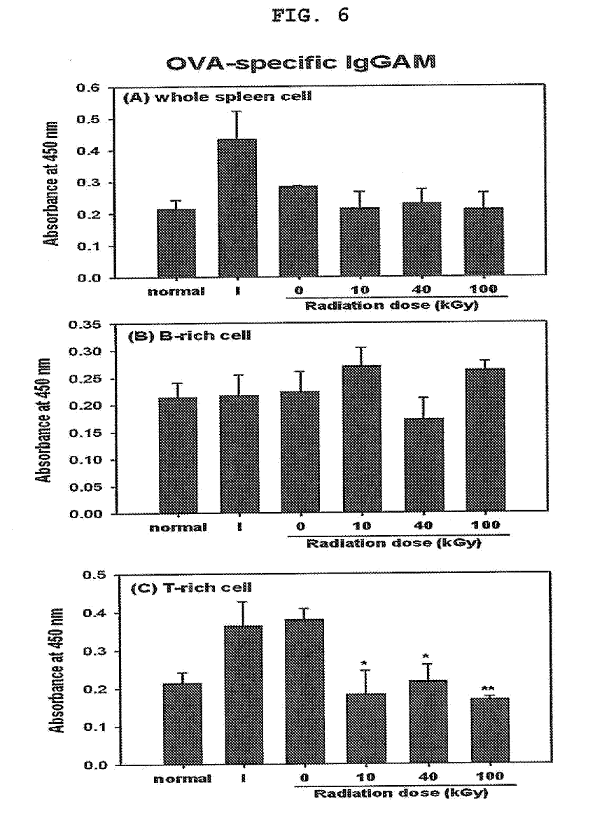

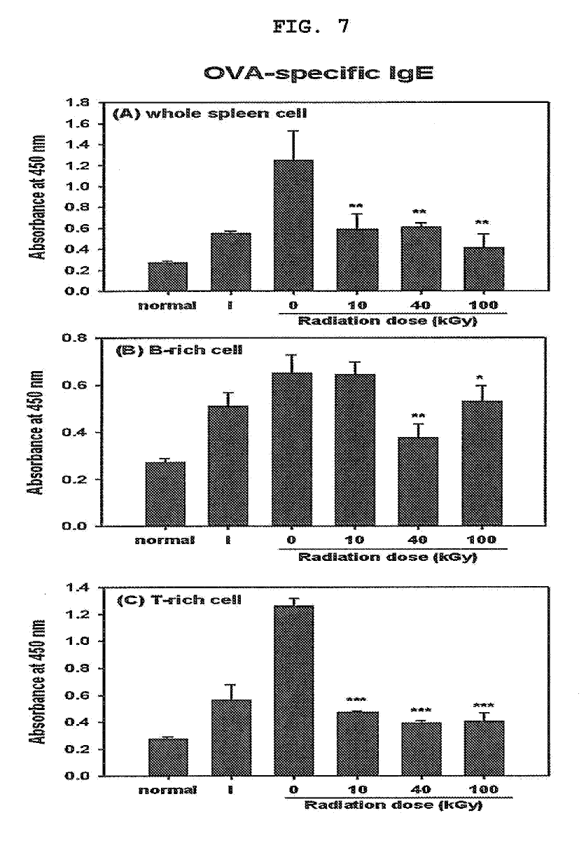

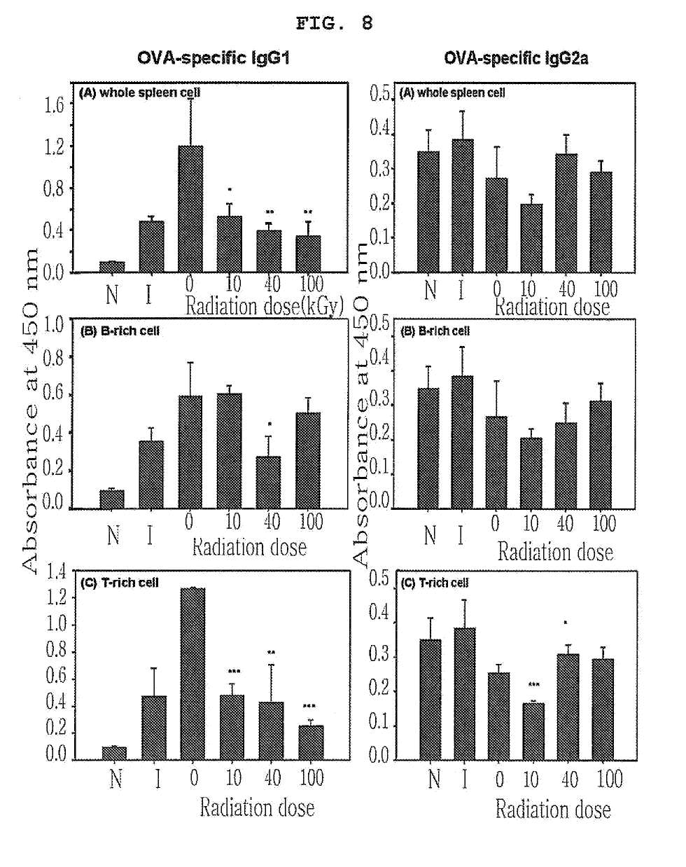

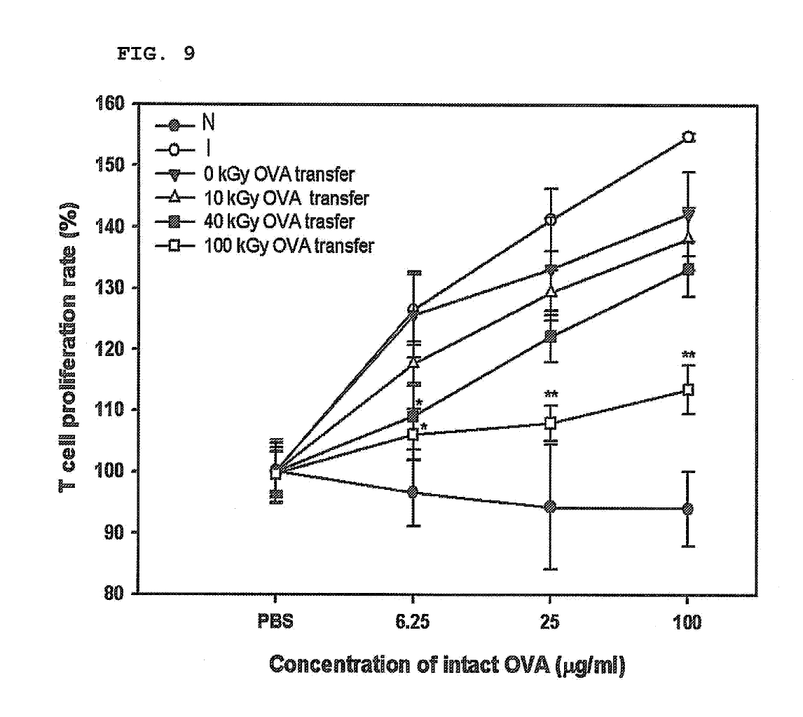

As a result, the levels of ovalbumin-specific IgGAM and ovalbumin-specific IgE antibody decreased radiation dose-dependently in the mouse vaccinated with the irradiated ovalbumin and over the radiation dose of 40 kGy, the levels were significantly decreased (see FIG. 2).

The ovalbumin-specific IgE can be used as an index for measuring allergic reaction and the decrease of ovalbumin-specific IgE in serum suggests the preventive effect by the irradiated ovalbumin and the effect was quite significant at the radiation dose of 40 kGy or higher.

The levels of such ovalbumin-specific IgG1, IgG2a, IgG2b, IgG3, IgM and IgA antibodies were also decreased radiation dose-dependently in serum of the mouse vaccinated with the irradiated ovalbumin (see FIG. 3).

The above results indicate that vaccination with the irradiated ovalbumin results in the decrease of ovalbumin-specific antibody production in relation to allergic reaction. These results also indicate that the vaccine composition of the present invention comprising the irradiated ovalbumin as an effective ingredient inhibits effectively the ovalbumin-specific humoral immune response induced by ovalbumin.

Second, the present inventors investigated the effect of the vaccine comprising the irradiated ovalbumin on cell mediated immune response.

As a result, the levels of Th1 response related cytokines IL-2 and IFN-γ and the levels of Th2 response related cytokines IL-4 and IL-10 were reduced in the mouse immunized with the irradiated ovalbumin (see FIG. 4). The inventors further investigated radiation dose-dependent T-cell proliferation in the mouse immunized with the irradiated ovalbumin (see FIG. 5).

The generation of the above cytokines and the decrease of T-cell proliferation indicate that the vaccination effect of the irradiated ovalbumin specifically on the cell mediated immune response caused by ovalbumin is great.

To confirm the above results shown in the mouse immunized with the irradiated ovalbumin, T-cells and B—cells were separated from the spleen of the mouse immunized with the irradiated ovalbumin, and then those separated cells were injected into a mouse not-treated to induce allergic reaction. Then, humoral immunity and cell mediated immunity of the mouse were investigated.

Humoral immune response and cell mediated immune response were reduced in the mouse transplanted with the spleen cells of the mouse immunized with the irradiated ovalbumin, which was consistent with the above results (see FIG. 6-FIG. 9 and Table 1). This result was more clearly confirmed in the mouse particularly transplanted with T-cells separated from the spleen of the mouse immunized with the irradiated ovalbumin.

The above results indicate that the vaccine composition for the prevention and treatment of allergic disease comprising the irradiated ovalbumin as an effective ingredient can reduce allergenicity even after allergic reaction is already induced. Therefore, the vaccine composition of the present invention can be effectively used for the prevention and treatment of allergic disease in particular food allergy.

The vaccine composition can be administered with a pharmaceutically or physiologically acceptable vehicle using saline or phosphate buffered saline or ethanol polyol such as glycerol or propylene glycol.

The vaccine composition of the present invention can additionally include an adjuvant selected from the group consisting of vegetable oil or its emulsion; surfactant such as hexadecylamine, octadecyl amino acid ester, octadecylamine, lysolecithin, dimethyl-dioctadecylammonium bromide, N,N-dioctadecyl-N′-N′bis(2-hydroxyethyl-propanediamine), methoxyhexadecylglycerol, and pluronic polyol; polyamine such as pyran, dextransulfate, poly IC, carbopol; peptide such as muramyl dipeptide, dimethylglycine and tuftsin, immunostimulating complex; oil emulsion; lipopolysaccharide such as MPLR (3-O-deacylated monophosphoryl lipid A; RIBI ImmunoChem Research, Inc., Hamilton, Mont.); and mineral gel.

The vaccine composition of the present invention can be administered by various pathways including parenteral administration, intra-arterial injection, intradermal injection, percutaneous insertion (by using sustained-releasing polymer), intramuscular injection, intra-abdominal injection, intravenous injection, hypodermic injection and intra-nasal insertion, but not always limited thereto.

The present invention further provides a method for the prevention and treatment of allergic disease using the vaccine composition of the invention.

The vaccine comprising the irradiated ovalbumin is preferably administered at 1-6 weeks intervals, 2-6 times, and more preferably administered at 1-3 weeks intervals, 2-4 times, and most preferably administered at one week intervals, twice. At this time, the administration is performed by parenteral or percutaneous administration.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation Method of the Irradiated Ovalbumin

### <1-1> Preparation of Ovalbumin

The allergen used in this invention was ovalbumin that is a major allergen of an egg.

Albumen and yolk were separated from an egg, and the albumen was 10-fold diluted in 0.01 M phosphate buffer (pH 6.5). Then insoluble impurities were eliminated by filtering the solution with a 0.45 μm filter. A 50×3 cm column was filled with DEAE (diethylamino ethyl) resin, followed by stabilization with 0.01 M phosphate buffer (pH 6.5). The albumen solution was loaded into the column, which was washed with the above buffer until other substances were not eluted. The protein adhered on the DEAE resin was eluted by salt density gradient method using the buffer comprising the above buffer and 0.4 M NaCl OD₂₈₀ of the eluted substance was measured using a UV spectrophotometer. The substance showing the peak was confirmed with electrophoresis and washed with 0.01 M phosphate buffer (pH 7.4), resulting in L the ovalbumin dissolved in 0.01 M phosphate buffer (pH 7.4) at the concentration of 1 mg/ml.

### <1-2> Ovalbumin Irradiation

The ovalbumin prepared in Example <1-1> was placed in a 1.5 ml tube and then the tube was covered with a lid. Irradiation was performed in Gamma ray irradiation laboratory, Advanced Radiation Technology Institute Jeongeup, Korea Atomic Energy Research Institute (radiation source: 1 million Ci, Co-60) at room temperature (10±0.5° C.) at the dose of 10 kGy/hour. Radiation dose of gamma ray was adjusted to make the total absorbed dose to be 0, 10, 40, and 100 kGy. The absorbed dose was checked by ceric-cerous dosimeter (Bruker Instruments, Rheinstetten, Germany) and the standard error of the total absorbed dose was t 0.1 kGy.

The resultant gamma ray irradiated ovalbumin was named as “irradiated ovalbumin”. All the experiments were repeated three times and mean value and standard error were calculated, which were used for two tailed student t-test with non-irradiated ovalbumin and irradiated ovalbumin. It was considered as statistically significant when P<0.05.

### EXAMPLE 2

### Preparation of the Mouse Treated with the Irradiated Ovalbumin

To evaluate the vaccination effect of the irradiated ovalbumin on allergic reaction, male BALE/c mice (Orientbio, Korea) at 6 weeks were purchased, followed by adaptation for one week and immunization.

20 μg of non-irradiated ovalbumin and 2.6 μg of aluminum hydroxide adjuvant (referred as “adjuvant” hereinafter) were mixed, which was intra-abdominally injected at 0 week and 1 week respectively, resulting in the preparation of the control group. 20 μg of irradiated ovalbumin and 2.6 mg of the adjuvant were mixed, which was intra-abdominally injected at 0 week and 1 week, resulting in the preparation of the experimental group. The radiation dose of gamma ray irradiated to the ovalbumin was, as described in Example <1-2>, 10, 40 and 100 kGy.

To induce allergic reaction, 20 μg of non-irradiated ovalbumin combined with the adjuvant was intra-abdominally injected to the control and experimental group mice after 2 weeks, 3 weeks and 4 weeks from the last immunization (FIG. 1).

To evaluate the vaccination effect of the irradiated ovalbumin, blood and spleen tissues were taken from the mice of each group after one week from the last induction. Blood samples and spleen tissues were also taken from the normal mouse group (non-treated, referred as group “N”) and from the mouse group with allergic reaction induced without immunization (referred as group “I”).

### EXPERIMENTAL EXAMPLE 1

### Evaluation of the Effect of the Vaccine Comprising the Irradiated Ovalbumin on Humoral Immunity

To confirm whether the vaccination with the irradiated ovalbumin, in Example 2, could inhibit allergic reaction induced by non-irradiated ovalbumin, humoral immunity was examined in the mice with allergic reaction induced after the vaccination. The examination of the humoral immune response was performed by enzyme immunoassay investigating ovalbumin specific antibody reaction in each mouse serum.

The non-irradiated ovalbumin (IgE; 10 μg/ml and IgG; 1 μg/ml) was placed on polystyrene flat-bottom microtiter plates (Maxisorp, Nunc, Kamstrup, Denmark) and fixed with 0.2 M bicarbonate buffer (pH 9.6) for overnight. After washing, the ovalbumin was reacted with 2% bovine serum albumin for one hour not to induce any other specific reaction. After washing, the mouse serum was diluted respectively with biotinylated IgE antibody (IgE-biotin, bioscience, USA) at the ratio of 1-20, with biotinylated IgG2a antibody (IgG2a-biotin, bioscience, USA) at the ratio of 1:100, with HPR (horseradish peroxidase) labeled IgGAM antibody (IgGAM-HRP, Southern biotech, USA), biotinylated IgG1 antibody (IgG1-biotin, bioscience, USA), biotinylated IgG2b antibody (IgG2b-blotin, BD bioscience, USA), biotinylated IgA antibody (IgA-biotin, BD bioscience, USA) and HPR-labeled IgM antibody (IgM-HRP, Southern biotech, USA) at the ratio of 1:1000, followed by reaction. After two hours of reaction, the ovalbumin was washed and reacted as follows. The secondary antibody for the biotinylated antibody was diluted in streptoavidin-HRP conjugate, (1:1000, BD bioscience, USA) and added to the ovalbumin, followed by reaction for one hour. The HRP-labeled antibody and streptoavidin-HRP conjugated antibody were washed and color development was induced using enzyme substrate solution (TMB, soluble, Calbiochem, USA) and then the reaction was terminated with 0.5 M H₂SO₄. The ovalbumin-specific antibody reaction in each group was investigated by measuring OD₄₅₀ using a microplate reader (Bio-Rad laboratories, USA).

As a result, when allergic reaction was induced with the noon-irradiated ovalbumin in the experimental group immunized with the irradiated ovalbumin, ovalbumin-specific IgGAM and ovalbumin-specific IgE antibody levels were reduced radiation dose-dependently, compared with the control group immunized with the non-irradiated ovalbumin and had allergic reaction induced by the non-irradiated ovalbumin. In particular, these antibody levels were significantly reduced at the radiation dose of 40 kGy and higher (FIG. 2).

The levels of such ovalbumin-specific IgG1, IgG2a, IgG2b, IgG3, IgM and IgA antibodies were also decreased radiation dose-dependently in the serum of the experimental group immunized with the irradiated ovalbumin (FIG. 3).

The above results indicate that vaccination by the irradiated ovalbumin results in the significant decrease of ovalbumin-specific antibody production by allergic reaction as a whole.

### EXPERIMENTAL EXAMPLE 2

### Evaluation of the Effect of the Vaccine Comprising the Irradiated Ovalbumin on Cell Mediated Immunity

### <2-1> Analysis of Cytokines Released from the Spleen Cells of the Mouse Immunized with the Irradiated Ovalbumin

To investigate cell-mediated immunity induced by the irradiated ovalbumin of the invention, spleen tissues were extracted from each mouse group as described in Example <1-2>. The spleen tissues were homogenized, leading to single cells. The cells were added into the complete medium prepared by adding 10% FES, 100 U/ml of penicillin and streptomycin to RPMI-1640 (Gibco, USA), which was inoculated into a 96 well plate (Falcon, USA) at the concentration of 1×10⁶ cells/well. The irradiated ovalbumin was added thereto (100 μg/ml) to resensitize. The cells were cultured for 72 hours in a 37° C. 5% CO₂ incubator and then the supernatant was obtained. Cytokines released from the spleen cells were analyzed. The Th1 and Th2 related cytokine level changes were measured according to the protocol of BD OptEIA™ mouse IL-2, IL-4, IL-10 and IFN-γ set (BD Bioscience, USA).

As a result, the levels of IL-2 and IFN-γ, involved in Th1 response, and the levels of IL-4 and IL-10, involved in Th2 response, were reduced in the mouse immunized with the irradiated ovalbumin (experimental group), compared with the mouse immunized with the non-irradiated ovalbumin (control group) (FIG. 4).

### <2-2> Proliferation of the Spleen Cells of the Mouse Immunized with the Irradiated Ovalbumin

After analyzing cytokine changes in Experimental Example <2-1>, the proliferation of the spleen cells of the mouse immunized with the irradiated ovalbumin was investigated. The proliferation of the spleen cells was measured by MTT [3-(4,5-dimethylthiazolyl)-2,5-diphenyl-tetrazolium bromide, Sigma].

MTT solution (5 μg/ml) was added to the spleen cells cultured for 72 hours at the concentration of 30 μl/well, followed by reaction for 2 hours in a 5% CO₂ incubator. Centrifugation was then performed to remove supernatant. Cell lysis was performed with 100 μl of DM50 (dimethysulfoxide, Sigma, USA) and OD₅₄₀ was measured.

As a result, T-cell proliferation of the mouse immunized with the irradiated ovalbumin (experimental group) was decreased radiation dose-dependently and the decrease of the T-cell proliferation was comparatively significant, compared with T-cell proliferation of the mouse immunized with non-irradiated ovalbumin (control group). The T-cell proliferation observed in the spleen cells of the mouse (I) with allergic reaction induced by ovalbumin was similar to the T-cell proliferation of the mouse immunized with non-irradiated ovalbumin (FIG. 5).

The above results indicate that the spleen cells immunized with the irradiated ovalbumin exhibit lower allergic response than the cells immunized with non-irradiated ovalbumin.

### EXPERIMENTAL EXAMPLE 3

### Evaluation of Humoral Immunity in Relation to the Decrease of Allergic Reaction in the Immune Cells Immunized with the Irradiated Ovalbumin

As shown in Example 2, the control group was immunized with non-irradiated ovalbumin but was not administered any other allergen. The experimental group was immunized with the irradiated ovalbumin and was not administered with any other allergen. Spleen cells were separated respectively from the normal mouse group (N) the mouse group with allergic reaction induced without immunization (I), the control group and the experimental group by the same manner as described in Example 2, and T-cells and B-cells were separated therefrom

T-cells were separated from the spleen cells using a nylon wool column.

The nylon wool column was washed with 37° C. RPMI (Gibco, USA) and filled with RPMI supplemented with warm PBS. The spleen cells were added to the column, followed by reaction for 45 minutes at 37° C. The column was eluted with 20 ml of warm RPMI and T-cells were collected in a tube for further experiments.

1×10⁷ spleen cells were conjugated in RPMI medium for 2 hours and cultured with anti-Thy 1.2 antibody (Cedarlane, Ontario, Canada). Then, the cells were cultured with the complement of a rabbit (Cedarlane) to hydrolyze all the cells except B-cells (mostly T-cells), followed by centrifugation to obtain B-cells only.

200 μl of phosphate buffer was added to the T-cells and B-cells separated as the above and spleen cells, resulting in cell suspension at the concentration of 1×10⁷. 1×10⁷ cells were intravenously injected under the tail of the allogenic BALBR/c mouse non-treated.

Ovalbumin-specific antibody titer was measured by the same manner as described in Experimental Example 1.

As a result, the levels of ovalbumin-specific IgGAM, ovalbumin-specific IgE and ovalbumin specific IgG1 were increased in the mouse transplanted with the spleen cells separated from the mouse immunized with non-irradiated ovalbumin. However, the change of the level of ovalbumin-specific IgG2a was not significant, suggesting that Th2 response was successfully induced, that is allergic reaction was successfully induced. The levels of ovalbumin-specific IgGAM, IgG1 and IgE of the mouse transplanted with the spleen cells, B-cells and T-cells separated from the mouse immunized with irradiated ovalbumin (experimental group) were much lower than those in the mouse immunized with non-irradiated ovalbumin (control group), and particularly the significant level drop was detected at the radiation dose of 10 kGy and higher (FIG. 6-FIG. 8). The level of ovalbumin-specific IgG2a was slightly reduced in T-cells of the mouse immunized with irradiated ovalbumin (10 kGy) but not significantly or rather increased in other groups, suggesting that Th2 response (allergic reaction) was inhibited.

The above results indicate that T-cells play an important role in inducing immune tolerance among T-cells, B-cells and spleen cells. This implication is supported by the result saying that the level of ovalbumin-specific IgE in the mouse transplanted with T-cells was similar to that in the mouse transplanted with spleen cells.

### EXPERIMENTAL EXAMPLE 4

### Evaluation of Cell Mediated Immunity in Relation to the Decrease of Allergic Reaction in the Immune Cells Immunized with the Irradiated Ovalbumin

As shown in Experimental Example 3, T-cells among the immune cells were confirmed to play an important role in inducing immune response in the mouse immunized with the irradiated ovalbumin. Thus, for the evaluation of cell mediated immunity, T-cell proliferation and cytokine production in the spleen cells of the mouse transplanted with T-cells were investigated.

As described in Experimental Example 3, T-cells alone were separated and injected intravenously into the allogenic BALB/c mouse. Then, allergic reaction was induced twice (on the next day and one week after the transplantation) by the same manner as described in Example 2. One week after the last induction of allergic reaction, spleen cells were separated and cultured for 72 hours with non-irradiated ovalbumin by the same manner as described in Experimental Example 3 and then T-cell proliferation and cytokine production were investigated.

As a result, T-Cell proliferation was very low in the mouse transplanted with T-cells of the mouse immunized with the irradiated ovalbumin (FIG. 9). This result was consistent with that of Experimental Example 2, which means T-cells separated from the spleen cells of the mouse immunized with the irradiated ovalbumin can inhibit T-cell proliferation.

The levels of cytokines from T-cells of the mouse transplanted with T-cells separated from the mouse immunized with the irradiated ovalbumin were measured one week after the induction of the last allergic reaction and the cytokines tested herein were IL-2, IFN-r, IL-2 and IFN-r.

As a result, even after inducing allergic reaction, Th1 (IL-2 and IFN-r) and Th2 response related cytokine (IL-2 and IFN-r) productions were reduced (Table 1), compared with those in T-cells of the mouse immunized with the irradiated ovalbumin, which was consistent with the result of Experimental Example 2.

As shown in the above results, vaccination with the irradiated ovalbumin inhibits allergic reaction even with the forced inducement of allergic reaction, and this inhibitory effect is radiation dose-dependent

TABLE 1
Comparison of cytokine productions in the spleen
cells of the allogenic mouse transplanted with T-cells
separated from the mouse immunized with the irradiated
ovalbumin

| | | | | Gamma ray irradiation | | |
|---|---|---|---|---|---|---|
| | N | I | O | 10 | 40 | 100 |
| IL-2 | 2.1 ± 1.8 | 30.8 ± 12.1 | 94.2 ± 8.9 | 112.3 ± 3.6 | 74.7 ± 8.9* | 67.6 ± 7.4* |
| IL-4 | 0 | 95.4 ± 5.2 | 179.7 ± 11.3 | 168.3 ± 3.4 | 96.8 ± 7.4* | 38.3 ± 1.5* |
| IL-6 | 123.5 ± 21.7 | 3303.5 ± 71.8 | 3858.5 ± 56.2 | 2513.5 ± 15.9* | 2318.5 ± 12.6* | 1818.5 ± 20.6* |
| IFN-γ | 0 | 188.7 ± 12.4 | 275.3 ± 24.1 | 402.0 ± 10.6* | 687.0 ± 7.5* | 68.7 ± 20.4** |

*p < 0.05;
**p < 0.01;
N: normal mouse group without treatment;
I: control group induced with allergic reaction without immunization;
O: spleen cells of the control group induced with allergic reaction after immunizing with non-irradiated ovalbumin;
10: spleen cells of the experimental group induced with allergic reaction after immunizing with irradiated ovalbumin at 10 kGy;
40: spleen cells of the experimental group induced with allergic reaction after immunizing with irradiated ovalbumin at 40 kGy; and
100: spleen cells of the experimental group induced with allergic reaction after immunizing with irradiated ovalbumin at 100 kGy.

The Manufacturing Example of the composition for the present invention is described hereinafter.

### MANUFACTURING EXAMPLE

### Preparation of Vaccine

Irradiated ovalbumin of the present invention 20 μg

Aluminum hydroxide 2.6 mg

Ovalbumin was conjugated to the final volume of aluminum hydroxide and the final volume was adjusted by using phosphate buffer (PO₄ 10 mM, NaCl 150 mM) to 1 ml per administration. The composition was stored at 4° C. until use.

### INDUSTRIAL APPLICABILITY

The irradiated ovalbumin prepared by irradiating the ovalbumin separated and purified from the albumen of an egg is a modified allergen. Humoral and cell mediated immune responses were all reduced in the mouse immunized with this irradiated ovalbumin, which suggests that the irradiated ovalbumin can inhibit allergic reaction. Thus, the composition of the present invention can be effectively used as a vaccine for the prevention and treatment of allergic disease.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for preparing all immunogen of a vaccine for the prevention and treatment of allergic disease, which comprises the following steps:
1) Separating and purifying ovalbumin from an egg; and
2) Irradiating the ovalbumin in aqueous solution.

2. The method for preparing an immunogen of a vaccine for the prevention and treatment of allergic disease according to claim 1, wherein the ovalbumin is a protein or glycoprotein that acts as an allergen being able to induce allergic reaction in human or animals.

3. The method for preparing an immunogen of a vaccine for the prevention and treatment of allergic disease according to claim 1, wherein the radioactive ray of step 2) is selected from the group consisting of gamma ray, electron beam (beta ray) and X-ray.

4. The method for preparing an immunogen of a vaccine for the prevention and treatment of allergic disease according to claim 1, wherein the radiation dose of the radioactive ray of step 2) is 5-100kGy.

5. The method for preparing an immunogen of a vaccine for the prevention and treatment of allergic disease according to claim 1 or claim 4, wherein the radiation dose of the radioactive ray of step 2) is 40-100 kGy.

6. The method for preparing an immunogen of a vaccine for the prevention and treatment of allergic disease according to claim 5, wherein the radiation dose of the radioactive ray of step 2) is b 100 kGy.

7. The method for preparing an immunogen of a vaccine for the prevention and treatment of allergic disease according to claim 1, wherein the aqueous solution of step 2) is phosphate buffer.

8. The method for preparing an immunogen of a vaccine for the prevention and treatment of allergic disease according to claim 1, wherein the allergic disease of step 2) is food allergy.

9. A vaccine composition for the prevention and treatment of allergic disease comprising the irradiated ovalbumin of claim 1 as an immunogen.

10. The vaccine composition according to claim 9, wherein the irradiated ovalbumin is conjugated to aluminum hydroxide adjuvant at the ratio weight of 1:100-200.

11. The vaccine composition according to claim 10, wherein the irradiated ovalbumin is conjugated to aluminum hydroxide adjuvant at the weight ratio of 1:100-150.

12. The vaccine composition according to claim 11 wherein the irradiated ovalbumin is conjugated to aluminum hydroxide adjuvant at the weight ratio of 1:130.

13. The vaccine composition according to claim 9, wherein the allergic disease is food allergy.

14. A method for the prevention and treatment of allergic disease using the vaccine composition of claim 9.

15. The method for the prevention and treatment of allergic disease according to claim 14, wherein the vaccine composition is administered at 1-6 weeks intervals, 2-6 times.

16. The method for the prevention and treatment of allergic disease according to claim 15, wherein the vaccine composition is administered at 1-3 weeks intervals, 2-4 time.

17. The method for the prevention and treatment of allergic disease according to claim 16, wherein the vaccine composition is administered at one week intervals, twice.

18. The method for the prevention and treatment of allergic disease according to claim 15, wherein the administration is performed by parenteral or percutaneous administration.

19. The method for the prevention and treatment of allergic disease according to claim 15, wherein the vaccine composition contains the irradiated ovalbumin at the concentration of 0.5-3 μg/g (weight).
